(19) United States
(12) Patent Application Publication
(10) Pub. No.: US 2024/0124209 A1
(43) Pub. Date: Apr. 18, 2024

(54) SUPPLEMENT CONTAINER AND METHOD OF USE FOR ANIMALS

(71) Applicant: PetSmart Home Office, Inc., Phoenix, AZ (US)

(72) Inventors: Zachary Parker Willard, Phoenix, AZ (US); Gregory J. Costanzo, Scottsdale, AZ (US)

(73) Assignee: PetSmart Home Office, Inc., Phoenix, AZ (US)

(21) Appl. No.: 17/964,394

(22) Filed: Oct. 12, 2022

(51) Int. Cl. B65D 81/32; A23K 20/174; A23K 20/24; A23K 40/30; B65D 21/02; B65D 51/18

(52) U.S. Cl. CPC B65D 81/3211; A23K 20/174; A23K 20/24; A23K 40/30; B65D 21/0228; B65D 51/18; A23K 10/20; B65D 2251/0021; B65D 2251/0081

(57) ABSTRACT

An apparatus for use with animals can include a first container comprising a first end and a second end, wherein the first end comprises a first fastener located on a first surface of the first container and the second end comprises a second fastener positioned on a second surface of the first container, and wherein the first surface is different form the second surface; a first lid comprising a third end and a fourth end, wherein fourth end comprises a third fastener located on a surface of the first lid, wherein the first lid is configured to be coupled to the first end of the first container by coupling together the third fastener and the first fastener; and a second container including a fifth end and a sixth end, wherein the fifth end comprises a fourth fastener located on a third surface of the second container, wherein the sixth end comprises a fifth fastener located on a fourth surface of the second container, wherein the third surface is different from the fourth surface, and wherein the second container is configured to be coupled to the second end of the first container by coupling together the fourth fastener and the second fastener. Other embodiments are disclosed.

### TECHNICAL FIELD

This disclosure relates generally to supplement containers and methods for using supplement containers.

### BACKGROUND

Animals that live in domestic locations can have difficulty obtaining the proper nutrition that they would obtain in the wild. For example, some animals may be fed crickets while living in a domestic location such as a house. These animals may need additional supplements to remain healthy. Accordingly, the crickets (or other food) can be coated with additional supplements to help keep the animals healthy.

### BRIEF DESCRIPTION OF THE DRAWINGS

To facilitate further description of the embodiments, the following drawings are provided in which:

FIG. 1A illustrates a front perspective, exploded view of a supplement container, according to an embodiment;

FIG. 1B illustrates a front perspective, assembled view of the supplement container of FIG. 1A;

FIG. 2A illustrates a cross-section along section line 250 of a first container of the supplement container in FIG. 1, according to an embodiment;

FIG. 2B illustrates an enlarged cross-section of a portion of the cross-section along section line 250 of the first container of the supplement container in FIG. 1, according to an embodiment;

FIG. 3A illustrates a cross section along section line 350 of a lid of the supplement container in FIG. 1, according to an embodiment;

FIG. 3B illustrates a bottom perspective view of a flap of the lid of the supplement container in FIG. 1, according to an embodiment;

FIG. 4A illustrates a cross section along section line 450 of a second lid of a second container of the supplement container in FIG. 1, according to an embodiment;

FIG. 4B illustrates a cross section along section line 470 of the second container of the supplement container in FIG. 1, according to an embodiment;

FIG. 5 illustrates a flow chart of a method for manufacturing a supplement container, according to an embodiment;

FIG. 6 illustrates a flow chart of a method for using a supplement container, according to an embodiment;

FIG. 7 illustrates a flow chart of a method for using a supplement container, according to an embodiment; and

FIG. 8 illustrates a flow chart of a method for using a supplement container, according to an embodiment.

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

The terms “couple,” “coupled,” “couples,” “coupling,” and the like should be broadly understood and refer to connecting two or more elements mechanically and/or otherwise. Coupling may be for any length of time, e.g., permanent or semi-permanent or only for an instant. The absence of the word “removably,” “removable,” and the like near the word “coupled,” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

### DESCRIPTION OF EXAMPLES OF EMBODIMENTS

Various embodiments are directed to an exemplary supplement container. The supplement container comprises a first container comprising a first end and a second end. In certain embodiments, the first end comprises a first fastener located on a surface of the first container and the second end comprises a second fastener positioned on the surface of the first container. In the certain embodiments, the surface can an external surface of the first container, and the first container also comprises an internal surface opposite the external surface.

In a number of embodiments, the second end of the first container comprises a structure comprising apertures. In certain embodiments, the structure is positioned on the internal surface of the first container adjacent to the second end of the first container. In certain embodiments, the apertures include a first size (or more than one first sizes) on a first side of the structure and a second size (or more than one second sizes) on a second side of the structure. In these certain embodiments, the structure of the apertures mitigates excess content from passing through the apertures, thereby reducing waste. In some of these certain embodiments, the first size(s) of the apertures at the first side of the structure is larger than the second size(s) of the apertures at the second side of the structure, and the first side of the structure faces inward towards a middle of the first container while the second side of the structure faces away from the middle of the first container. In some embodiments, the structure can be a sieve. In some embodiments, the apertures of the structure are diamond shaped.

In certain embodiments, the first end of the first container comprises a first diameter, and the second end of the first container comprises a second diameter. In some embodiments, the first diameter is different than the second diameter. For example, the first diameter can be larger than the second diameter. In certain embodiments, the first container includes a third diameter adjacent to a top surface of the structure comprising the apertures. In some embodiments, the first diameter, the second diameter, and the third diameter are sized to direct the flow of food and/or liquid from the first end to the second end in an efficient manner to mitigate the loss of food and/or liquid during operation. For example, the second diameter is sized to position the second end within a second container so that the second container can collect any excess food and/or liquid. In this example, the first diameter can be larger than the third diameter, which can be larger than the second diameter.

In certain embodiments, the supplement container comprises a lid comprising a third end and a fourth end. In certain embodiments, the fourth end comprises a third fastener located on a surface of the lid. In some embodiments, the lid is adapted to be coupled to the first end of the first container by coupling together the third fastener and the first fastener. In some embodiments, the surface can be an internal surface of the lid. In some embodiments, the third fastener can be located on an external surface of the lid, and the first fastener of the first container can be positioned on the internal surface of the first container. In such an embodiment, the third fastener can be coupled to the first fastener on the internal surface of the first container. In some embodiments, the third fastener of the lid can be positioned between the third end and the fourth end of the lid. In some embodiments, the third fastener can be positioned on or at the fourth end of the lid.

In certain embodiments, the third end of the lid comprises a first protrusion, a flap comprising a second protrusion, and a hinge. In some embodiments, the flap pivots about the hinge to allow access to an internal portion of the first container when the lid is coupled to the first container. In some embodiments, the first protrusion and the second protrusion can be located and sized to create a snap-fit connection with each other to mitigate the flap from moving during operation of the supplement container. In certain embodiments, the first fastener of the first container and the third fastener of the lid comprise threaded fasteners that couple together with each other to couple together the lid and the first container. In certain embodiments, the flap can include one or more notches that interact with openings on the hinge to couple the flap to the hinge. The lid also can include a recess to allow a user to operate the flap between an open and closed position. That is, the recess can creates a gap to allow a user to operate the lid with minimal effort, while still maintaining a tight seal for the flap during operation.

In certain embodiments, the third end of the lid and the fourth end of the lid comprise a third diameter. The third diameter can be sized to create a seal with the first end of the first container when the lid is coupled to the first container. In particular, the lid can be sized to create a seal with a first annular shoulder of the first container.

In certain embodiments, the second protrusion of the flap comprises a notch that comprises a lip. In certain embodiments, the notch and the lip can be sized to abut the first protrusion of the lid to maintain a seal with the lid. For example, the notch and the lip can be biased inwards by the first protrusion when a user operates the lid to a closed position until the lip passes the first protrusion and the notch abuts the first protrusion, thereby locking the flap in the closed position. In a similar manner, a user can lift the flap to disengage the notch and the lip of the flap from the first protrusion of the lid.

In certain embodiments, the supplement container comprises a second container including a fifth end and a sixth end. In certain embodiments, the fifth end comprises a fourth fastener located on a third surface of the second container, and the sixth end comprises a fifth fastener located on a fourth surface of the second container. In certain embodiments, the third surface is different from the fourth surface. In particular, the third surface can be an internal surface, and the fourth surface can be an external surface. In certain embodiments, the second container can be adapted to be coupled to the second end of the first container by coupling together the fourth fastener and the second fastener.

In certain embodiments, the second container comprises walls defining a cavity. In some embodiments, the walls comprise the third surface. In certain embodiments, the second container comprises a second lid. The second lid can be sized to create a friction fit with the walls to seal the cavity. In some embodiments, when the second lid is not sealing the cavity and when the second container is coupled to the second end of the first container, contents within the first container can pass through the apertures of the structure to enter the cavity to mitigate loss of the content (e.g., liquid, food, supplement, etc.).

In certain embodiments, the second lid can include a cavity and a handle. In some embodiments, when the second lid is sealing the cavity of the second container and when the second container is coupled to the second end of the first container, contents within the second container cannot pass through the apertures of the structure and can be maintained within the cavity of the second container, thereby mitigating the mixing of different contents in the first and second containers.

In other embodiments, a user can subsequently remove the second container from the first container, access the handle to remove the second lid from the second container, and re-couple the second container (without the second lid) to the first container. Now, the contents within the second container can pass through the apertures of the structure and mix with food or other contents in the cavity of the first container.

In certain embodiments, the fifth end of the second container comprises a fourth diameter, and the sixth end of the second container comprises a fifth diameter. In this embodiment, the fourth diameter can be different than the fifth diameter so that the fifth end can maintain a seal with a second annular shoulder of the first container and so that the sixth end can maintain a seal with a third container that is similarly sized as the second container. For example, the fourth diameter can be sized to create a seal with the first container when the fifth end of the second container is coupled to the second end of the first container.

In certain embodiments, the supplement container comprises a third container adapted to be coupled to the sixth end of the second container. In these embodiments, the third container can comprise a recess on an external surface of the third container. In some embodiments, the recess can be configured to receive a label to identify the contents of the third container. In some embodiments, a user can write in the recess with a writing utensil and/or other marking utensil. In these embodiments, the third container also can comprises a lid. The third container can operate in a similar manner to the second container. In some embodiments, the third container and the second container are identical to each other. In other embodiments, the third container includes the recess while the second container does not include a recess. The third container can be interchangeable with the second container such that the third container can be coupled directly to the second end of the first container while the second container is coupled directly to the third container.

In embodiments disclosed herein, the supplement container can be manufactured via injection molding. However, the supplement container can be manufactured via other types of manufacturing, as well, such as additive manufacturing. In some embodiments, the supplement container is comprised of a transparent static-free material. A transparent static-free material is utilized to mitigate food and other contents from sticking to the sides of the supplement container and to allow a user to see within the supplement container without opening the flap of the lid.

During operation, the first container can be configured to contain food for an animal. In some examples, the food can be crickets. In some embodiments, the second container can be configured to contain at least one of a vitamin or a calcium supplement, and the at least one of the vitamin or the calcium supplement can pass from the second container into the first container to coat the food when the second lid is not coupled to the second container and when the second container is coupled to the first container. In many embodiments, the vitamin can be a multivitamin. In some embodiments, after the vitamin or the calcium supplement has been passed from the second container to the first container, as described above, the second container can be coupled to the first container as detailed above to collect any excess amount of the vitamin or the calcium supplement that does not adhere to the food in the first container because such vitamin or calcium supplement can pass back through the apertures in the structure and into the second container. This configuration can reduce waste of the vitamin or the calcium supplement. In some embodiments, the opening of the lid can be sized to receive tweezers to allow a user to easily access the contents of the first container without the use of a tube or smaller device that current solutions utilize to get the food out of the first container, thereby improving access to the contents of the first container.

In various embodiments, a method of manufacturing an apparatus is disclosed. In certain embodiments, the method comprises: providing a first container, the first container comprising a first end and a second end, wherein the first end comprises a first fastener located on a first surface of the first container and the second end comprises a second fastener positioned on a second surface of the first container, and wherein the first surface is different form the second surface; providing a first lid, the first lid comprising a third end and a fourth end, wherein the fourth end comprises a third fastener located on a surface of the first lid, wherein the first lid is configured to be coupled to the first end of the first container by coupling together the third fastener and the first fastener; and providing a second container, the second container including a fifth end and a sixth end, wherein the fifth end comprises a fourth fastener located on a third surface of the second container, wherein the sixth end comprises a fifth fastener located on a fourth surface of the second container, wherein the third surface is different from the fourth surface, and wherein the second container is configured to be coupled to the second end of the first container by coupling together the fourth fastener and the second fastener.

In various embodiments, a method of using a supplement container is disclosed. In certain embodiments, the method comprises: coupling a second lid to a second container; coupling the second container to a second end of a first container while the second lid is coupled to the second container; inserting first food into the first container through a first end of the first container while the second container is coupled to the second end of the first container; inserting a first supplement into the first container through the first end of the first container while the second container is coupled to the second end of the first container; mixing the first food and the first supplement together in the first container to coat the first food with the first supplement, such that an excess amount of the first supplement passes through the second end of the first container onto the second lid coupled to the second container, which is coupled to the second end of the first container; removing the first food, as coated with the first supplement, from the first container; and removing the second container from the second end of the first container with the excess amount of the first supplement on the second lid.

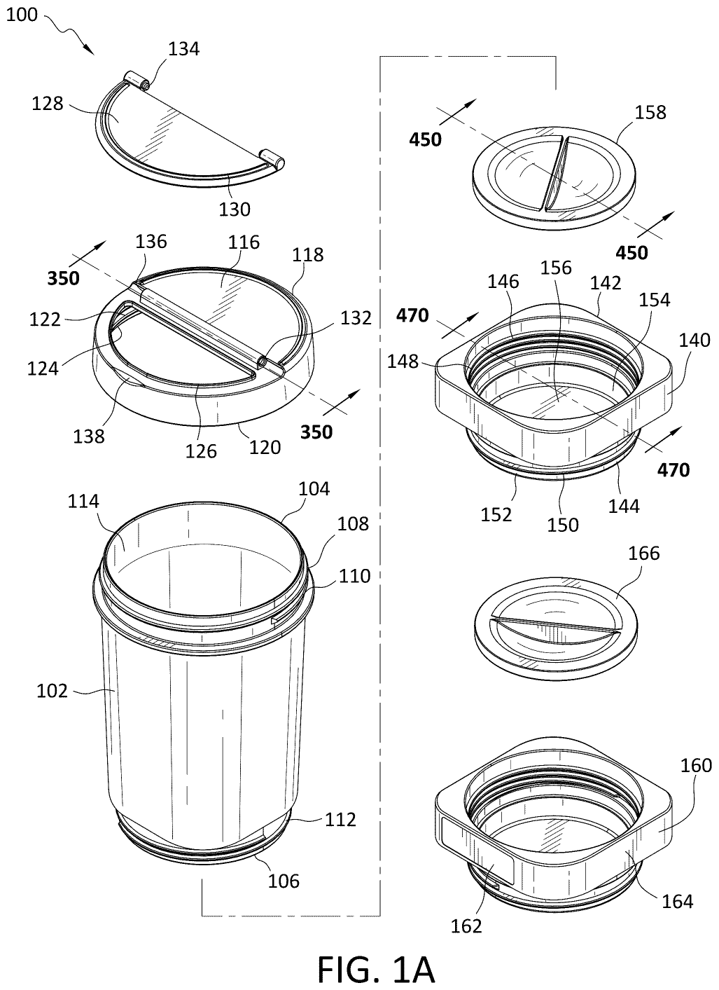

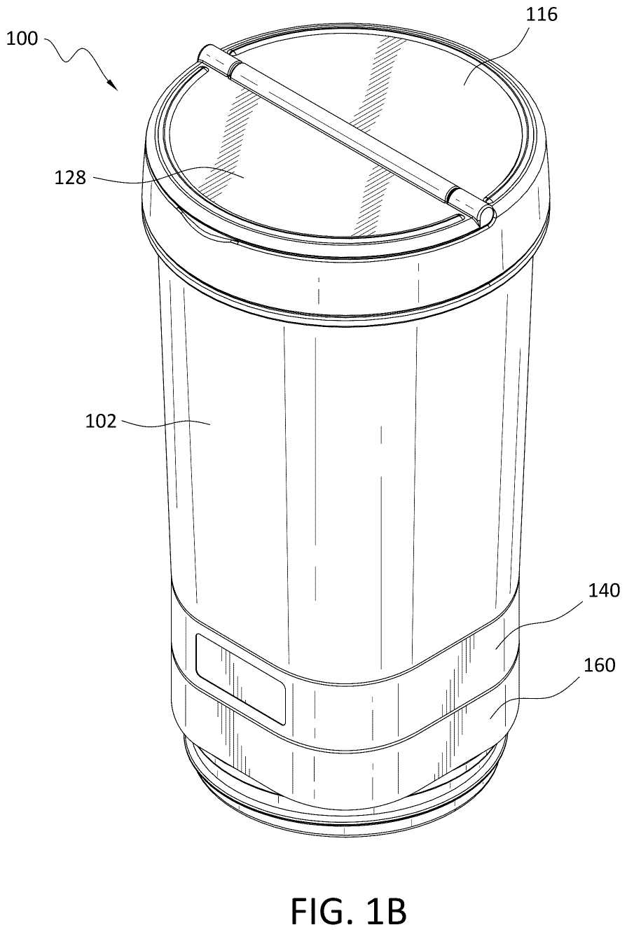

Turning to the drawings, FIG. 1 illustrates an exemplary supplement container 100. The supplement container 100 of the illustrated embodiment comprises a first container 102 comprising a first end 104 and a second end 106. In the illustrated embodiment, the first end 104 comprises a first fastener 108 located on a surface 110 of the first container 102, and the second end 106 comprises a second fastener 112 positioned on the surface 110 of the first container 102. In the illustrated embodiment, the surface 110 is an external surface, and the first container 102 comprises an internal surface 114.

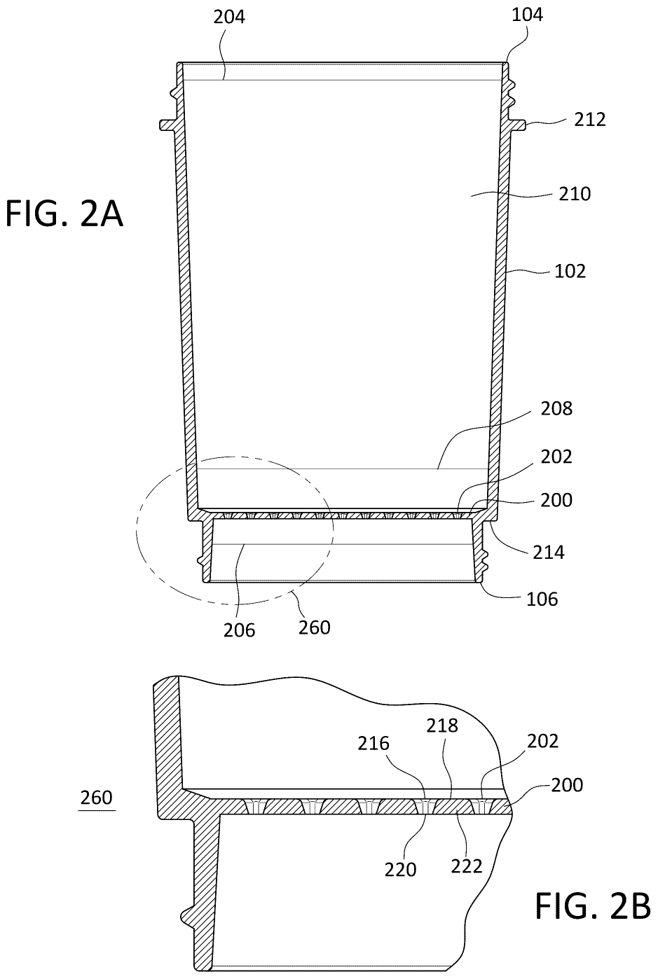

Turning briefly to FIG. 2A, in a number of embodiments, the second end 106 of the first container 102 comprises a structure 200 comprising apertures 202. Turning briefly to FIG. 2B, an exploded view of the cross section of the apertures 202 is illustrated. In the illustrated embodiment, the apertures 202 include a first size 216 on a first side 218 of the structure 200 and a second size 220 on a second side 222 of the structure 200. As an example each of first size 216 and second size 220 can be a diameter of opposite ends of an aperture through structure 200. In the illustrated embodiment, first size 216 is larger than second size 220 so that the structure of the apertures 202 mitigates excess content from passing through the apertures 202, thereby reducing waste. In some embodiments, the structure 200 is a sieve. In some embodiments, the apertures 202 of the structure 200 are diamond shaped. However, the apertures can be rectangular, triangular, trapezoid, elliptical, etc. In certain embodiments, size 216 of the apertures 202 can have a size of greater than 2.5 millimeters (mm), and size 220 of the apertures 202 can have a size of less than 4.75 mm. In some embodiments, size 216 is always greater than size 220.

In the illustrated embodiment of FIG. 2A, the first end 104 of the first container 102 comprises a first diameter 204, and the second end 106 of the first container 102 comprises a second diameter 206. In some embodiments, the first diameter 204 is different than the second diameter 206. In certain embodiments, the first diameter 204 can have a size of 50 mm-100 mm, and the second diameter 206 can have a size of 30 mm-90 mm. Referring back to FIG. 2A, the first container 102 includes a third diameter 208 adjacent to a top surface of the structure 200. In certain embodiments, the third diameter 208 can have a respective size of 35 mm to 95 mm. In some embodiments, the first diameter 204, the second diameter 206, and the third diameter 208 are sized to direct the flow of food and/or liquid from the first end 104 to the second end 106 in an efficient manner to mitigate the loss of food and/or liquid during operation, such as when food is being coated in the first container 102. For example, the second diameter 206 is sized to position the second end 106 within a second container (e.g., second container 140 of FIG. 1) so that the second container can collect any excess food, supplement, and/or liquid from the cavity of the first container 102 after the original contents of the cavity of the first container 102 have been coated with such food, supplement, and/or liquid.

Returning to FIG. 1, the supplement container 100 of the illustrated embodiment comprises a lid 116 comprising a third end 118 and a fourth end 120. In the illustrated embodiment, the fourth end 120 comprises a third fastener 122 located on a surface 124 of the lid 116. In some embodiments, the lid 116 can be configured to be coupled to the first end 104 of the first container 102 by coupling together the third fastener 122 and the first fastener 108. In some embodiments, the surface 124 can be an internal surface of the lid 116. In other embodiments (not shown in FIG. 1), the third fastener 122 can be located on an external surface of the lid 116, and the first fastener 108 is positioned on the internal surface 114 of the first container 102. In such an embodiment, the third fastener 122 can be coupled to the first fastener 108 on the internal surface 114 of the first container 102. In some embodiments, the third fastener 122 can be positioned between the third end 118 and the fourth end 120. In some embodiments, the third fastener 122 can be positioned on or at the fourth end 120.

In the illustrated embodiment, the third end 118 of the lid 116 comprises a first protrusion 126, a flap 128 comprising a second protrusion 130, and a hinge 132. In some embodiments, the flap 128 pivots about the hinge 132 to allow access to a cavity or an internal portion 210 (FIG. 2) of the first container 102 when the lid 116 is coupled to the first container 102. In some embodiments, the first protrusion 126 and the second protrusion 130 are located and sized to create a snap-fit connection with each other to mitigate the flap 128 from moving during operation of the supplement container 100. In the illustrated embodiments, the first fastener 108 of the first container 102 and the third fastener 122 of the lid comprise threaded fasteners that couple together with each other to couple together the lid 116 and the first container 102. In the illustrated embodiment, the flap 128 includes notches 134 that interact with openings 136 on the hinge 132 to couple the flap 128 to the hinge 132. The lid 116 also includes a recess 138 to allow a user to operate the flap 128 between an open and closed position. That is, the recess 138 creates a gap to allow a user to operate the lid 116 with minimal effort, while still maintaining a tight seal for the flap 128 during operation.

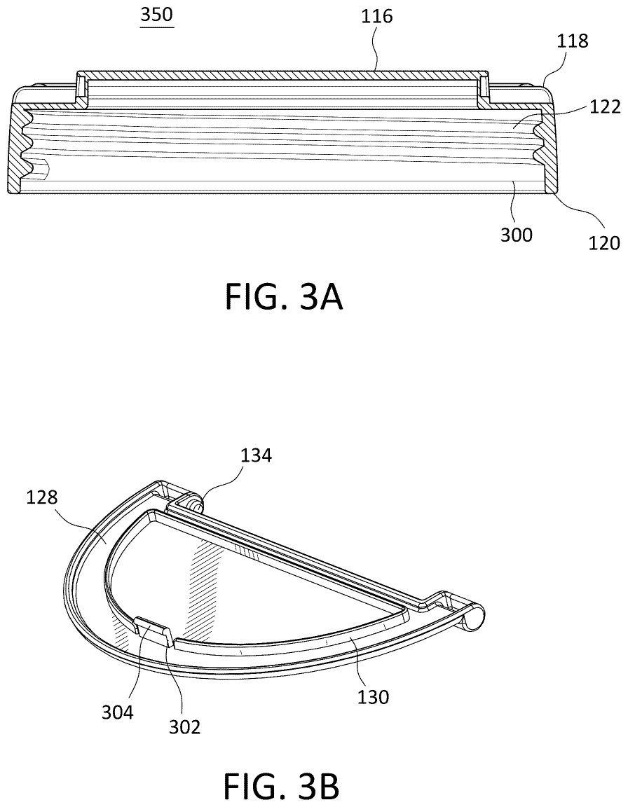

Turning briefly to FIG. 3A, the third end 118 of the lid 116 and the fourth end 120 of the lid 116 comprise a third diameter 300. In certain embodiments, the third diameter 300 can have a respective size of 55 mm-125 mm. The third diameter 300 is sized to create a seal with the first end 104 (FIG. 2A) of the first container 102 (FIG. 2A) when the lid 116 is coupled to the first container 102 (FIG. 2A). In particular, the lid 116 is sized to create a seal with the first annular shoulder 212 (FIG. 2A) of first container 102.

Turning to FIG. 3B, the second protrusion 130 of the flap 128 comprises a notch 302 that comprises a lip 304. In the illustrated embodiment, the notch 302 and the lip 304 are sized to abut the first protrusion 126 to maintain a seal with the lid 116 (FIG. 1A). For example, the notch 302 and the lip 304 are biased inwards by the first protrusion 126 (FIG. 1A) when a user operates the lid 116 (FIG. 1A) to a closed position (e.g., the flap 128 covers the internal portion 210 (FIG. 2A)) until the lip 304 passes the first protrusion 126 (FIG. 1A) and the notch 302 abuts the first protrusion 126 (FIG. 1A), thereby locking the flap 128 in the closed position. In a similar manner, a user lifts the flap 128 to disengage the notch 302 and the lip 304 from the first protrusion 126 (FIG. 1A).

Returning to FIGS. 1A and 1B, the supplement container 100 comprises a second container 140 including a fifth end 142 and a sixth end 144. In the illustrated embodiment, the fifth end 142 comprises a fourth fastener 146 located on a third surface 148 of the second container 140, and the sixth end 144 comprises a fifth fastener 150 located on a fourth surface 152 of the second container 140. In the illustrated embodiments, the third surface 148 is different from the fourth surface 152. In particular, the third surface 148 is an internal surface, and the fourth surface 152 is an external surface. In the illustrated embodiments, the second container 140 is adapted to be coupled to the second end 106 of the first container 102 by coupling together the fourth fastener 146 and the second fastener 112.

In the illustrated embodiments, the second container 140 comprises walls 154 defining a cavity 156. In some embodiment, the walls 154 comprise the third surface 148. In the illustrated embodiments, the second container 140 comprises a second lid 158. The second lid 158 is sized to create a friction fit with the walls 154 to seal the cavity 156. In some embodiments, the second lid 158 is sized between a range of 30 mm-90 mm. In some embodiments, when the second lid 158 is not sealing the cavity 156 and when the second container 140 is coupled to the second end 106 of the first container 102, contents within the first container 102 can pass through the apertures 202 (FIG. 2B) of the structure 200 (FIG. 2B) to enter the cavity 156 to mitigate loss of content (e.g., liquid, food, etc.) as it passes from the second container 140 to the first container 102.

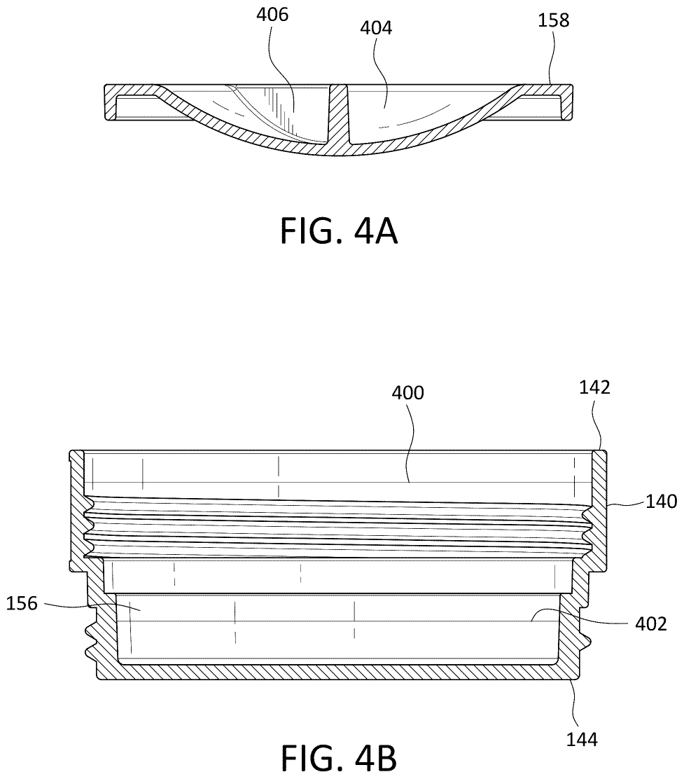

Turning to FIG. 4A, the second lid 158 includes a cavity 404 and a handle 406. In some embodiments, referring to FIGS. 4A and 4B, when the second lid 158 is not sealing the cavity 156 of the second container 140 and when the second container 140 is coupled to the second end 106 (FIG. 2A) of the first container 102 (FIG. 2A), contents within the first container 102 can pass through the apertures 202 (FIG. 2A) of the structure 200 (FIG. 2A) and can be maintained within the cavity 404 of the lid 158 instead of the cavity 156 of the second container 140, thereby mitigating the mixing of the contents from cavity 156 in cavity or inner portion 210 (FIG. 2A). In the illustrated embodiment, a user can access the handle 406 to remove the lid 158 from the second container 140 when the second container 140 is not coupled to the second end 106 (FIG. 1A) of the first container 102 (FIG. 1A).

Turning to FIG. 4B, the fifth end 142 of the second container 140 comprises a fourth diameter 400, and the sixth end 144 of the second container 140 comprises a fifth diameter 402. In the illustrated embodiment, the fourth diameter 400 can be different than the fifth diameter 402. In certain embodiments, the fourth diameter 400 can have a dimension of 30 mm-90 mm, and the fifth diameter 402 can have a dimension of 25 mm-85 mm. The fourth diameter 400 can be different than the fifth diameter 402 so that the fifth end 142 can maintain a seal with a second annular shoulder 214 (FIG. 2) of the first container 102 (FIG. 2) and so that the sixth end 144 can maintain a seal with a third container (e.g., third container 160 (FIG. 1A)) when the third container is the same or similar size as the second container 140. For example, the fourth diameter 400 is sized to create a seal with the first container 102 when the second container 140 is coupled to the first container 102.

Returning to FIG. 1A, the supplement container 100 comprises a third container 160 adapted to be coupled to the sixth end 144 of the second container 140. In the illustrated embodiment, the third container 160 can comprise a recess 162 on an external surface 164 of the third container 160. In some embodiments, the recess 162 can be configured to receive a label to identify the contents of the third container 160. In some embodiments, a user can write in the recess 162 with a writing utensil and/or other marking utensil. In the illustrated embodiment, the third container 160 comprises a lid 166. The third container 160 can operate in a similar manner to the second container 140. In some embodiments, the third container 160 and the second container 140 are identical to each other. In other embodiments, the third container 160 includes the recess 162 while the second container 140 not include such a recess. In the embodiment shown in FIG. 1B, the second end 106 of the first container 102 is coupled directly to the second container 140, which is coupled directly to the third container 160. In many embodiments, the third container 160 can be interchangeable with the second container 140 such that the second end 106 of the first container 102 can be coupled directly to the third container 160, which can be coupled directly to the second container 140.

In some embodiments, the first container 102, the second container 140, and the third container 160 can be different shapes. For example, first container 102, the second container 140, and the third container 160 can be circular, rectangular, triangular, etc. In some embodiments, the first container 102 can be circular, and the first and second containers 140, 160 can be rectangular.

In embodiments disclosed herein, the supplement container 100 is manufactured via injection molding. However, the supplement container 100 can be manufactured via other types of manufacturing, such as additive manufacturing or blow molding. In some embodiments, the supplement container 100 is comprised of a transparent static-free material, such as Acrylonitrile business system (ABS) with and anti-static agent additive. A transparent static-free material is utilized to mitigate food and other contents from sticking to the sides of the supplement container 100 and to allow a user to see within the supplement container 100 without opening the flap 128.

During operation, the first container 102 can be configured to contain food for an animal. In some example, the food can be crickets. In some embodiments, the second container 140 is adapted to contain at least one of a vitamin or a calcium supplement, and the at least one of the vitamin or the calcium supplement can pass from the second container 140 into the first container 102 to coat the food. In some embodiments, after the vitamin or the calcium supplement has been passed from the second container 140 to the first container 102, as described above, the second container 140 can be coupled to the first container 102 as detailed above to collect any excess amount of the vitamin or the calcium supplement that does not adhere to the food in the first container 102 because such vitamin or calcium supplement can pass back through the apertures 202 (FIG. 2A) in the structure 200 (FIG. 2A) and into the second container 140. This configuration can reduce waste of the vitamin or the calcium supplement. In some embodiments, the opening of the lid 116 can be sized to receive tweezers to allow a user to easily access the contents of the first container 102 without the use of a tube or smaller device that current solutions utilize to get the food out of the first container 102, thereby improving access to the contents of the first container 102.

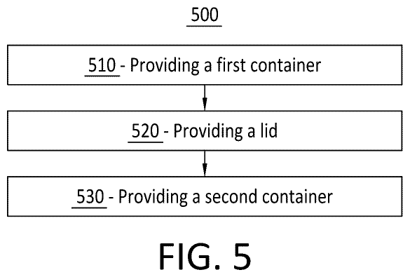

Turning ahead in the drawings, FIG. 5 illustrates a flow chart of a method 500 for manufacturing a supplement container 100 (FIGS. 1A-1B), according to an embodiment. In many embodiments, the method 500 can include: providing a first container (block 510), providing a lid (block 520), and providing a second container (block 530). In some embodiments, the method 500 for manufacturing the supplement container 100 (FIGS. 1A-1B) can include: providing a first container via injection molding, providing a lid via injection molding, and providing a second container via injection molding. In other embodiments, blocks 510, 520, and/or 530 can use other types of manufacturing processes. Examples of other types of manufacturing processes include extruding, casting, thermoforming, CNC (computer numerical control) machining, additive manufacturing such as 3-dimensional printing, and other types of molding such as blow molding, rotational molding, expanded bead foam molding, and extruded foam molding.

In a number of embodiments, the first container provided in block 510 can be similar or identical to the first container 102 (FIGS. 1A-1B) disclosed herein. For example, referencing FIGS. 1A-1B, the first container can include the first end 104 and the second end 106. The first end 104 can include the first fastener 108 located on the surface 110 of the first container 102, and the second end 106 comprises the second fastener 112 positioned on the surface 110 of the first container 102. In some embodiments, the surface 110 is the external surface, and the first container 102 comprises the internal surface 114. In some embodiments, providing the first container 102 further comprises providing the structure 200 (FIG. 2A) comprising the apertures 202 (FIG. 2A) on the second end 106 of the first container 102.

Returning to FIG. 5, in a number of embodiments, the lid in block 520 can be similar or identical to the lid 116 (FIGS. 1A-1B) disclosed herein. For example, referencing FIGS. 1A-1B, the lid can include the third end 118 and the fourth end 120. The fourth end 120 can include the third fastener 122 located on the surface 124 of the lid 116. In some embodiments, the lid 116 can be configured to be coupled to the first end 104 of the first container 102 by coupling together the third fastener 122 and the first fastener 108. In some embodiments, the first fastener 108 of the first container 102 and the third fastener 122 of the lid 116 comprise threaded fasteners that couple together with each other to couple together the lid 116 and the first container 102.

Returning again to FIG. 5, in a number of embodiments, the second container in block 530 can be similar or identical to the second container 140 (FIGS. 1A-1B). For example, referencing FIGS. 1A-1B, the second container can include the fifth end 142 and the sixth end 144. The fifth end 142 can include the fourth fastener 146 located on the third surface 148 of the second container 140, and the sixth end 144 can include the fifth fastener 150 located on the fourth surface 152 of the second container 140. In some embodiments, the third surface 148 is different from the fourth surface 152. In particular, the third surface 148 is an internal surface, and the fourth surface 152 is an external surface. In some embodiments, the second container 140 is configured to be coupled to the second end 106 of the first container 102 by coupling together the fourth fastener 146 and the second fastener 112. In some embodiments, providing the second container 140 further comprises providing the walls 154 defining the cavity 156. In some embodiments, the walls 154 comprise the third surface 148. In some embodiments, the second container 140 comprises a second lid 158. The second lid 158 is sized to create a friction fit with the walls 154 to seal the cavity 156. In some embodiments, the method 500 can provide a third container. In a number of embodiments, the third container can be similar or identical to the third container 160 (FIGS. 1A-1B). For example, the third container 160 is configured to be coupled to the sixth end 144 of the second container 140.

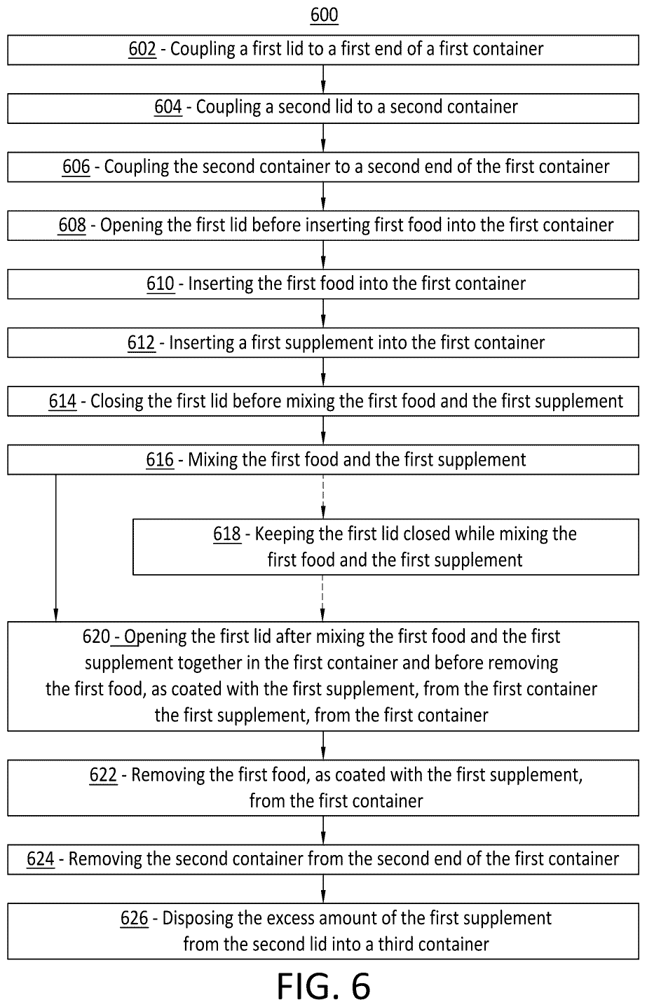

Turning ahead in the drawings, FIG. 6 illustrates a flowchart of a method (600) for using a supplement container (e.g., 100 (FIGS. 1A-1B)), according to an embodiment. As an example, method 600 can be used to coat food. In a number of embodiments, the supplement container of method 600 can be similar or identical to the supplement container 100 (FIGS. 1A-1B).

In a number of embodiments, the method 600 can include coupling a first lid to the first end of the first container (block 602). For example, block 602 can include coupling the lid 116 (FIGS. 1A-1B) to the first end 104 (FIGS. 1A-1B) of the first container 102 (FIGS. 1A-1B). In some embodiments, the coupling can include screwing or twisting the lid 116 (FIGS. 1A-1B) onto the first end 104 (FIGS. 1A-1B) of the first container 102 (FIGS. 1A-1B). In other embodiments, the coupling can include a snap-on coupling, a smooth friction fit, or the like.

In a number of embodiments, the method 600 also can include coupling a second lid to a second container (block 604). For example, block 604 can include coupling the second lid 158 (FIGS. 1A-1B) to the second container 140 (FIGS. 1A-1B). In some embodiments, the coupling can include pushing the second lid 158 (FIGS. 1A-1B) onto the second container 140 (FIGS. 1A-1B) to secure the second lid 158 (FIGS. 1A-1B) onto the second container 140 (FIGS. 1A-1B) via a smooth friction fit. In other embodiments, the coupling can include a snap-on coupling, a screwing or twisting motion, or the like.

In a number of embodiments, the method 600 further can include coupling the second container to a second end of the first container (block 606). For example, block 606 can include coupling the second container 140 (FIGS. 1A-1B) to the second end 106 (FIGS. 1A-1B) of the first container 102 (FIGS. 1A-1B) while the second lid 158 (FIGS. 1A-1B) is coupled to the second container 140 (FIGS. 1A-1B). In some embodiments, the coupling can include screwing or twisting the second container 140 (FIGS. 1A-1B) onto the second end 106 (FIGS. 1A-1B) of the first container 102 (FIGS. 1A-1B). In other embodiments, the coupling can include a snap-on coupling, a smooth friction fit, or the like.

In a number of embodiments, the method 600 additionally can include opening the first lid before inserting first food into the first container (block 608). For example, block 608 can include opening the flap 128 (FIGS. 1A-1B) of the lid 116 (FIGS. 1A-1B) while the lid 116 (FIGS. 1A-1B) is coupled to the first end 104 (FIGS. 1A-1B) of the first container 102 (FIGS. 1A-1B).

In a number of embodiments, the method 600 also can include inserting the first food into the first container (block 610). For example, block 610 can include inserting first food (e.g., crickets) into the first container 102 (FIGS. 1A-1B) through the first end 104 (FIGS. 1A-1B) of the first container 102 (FIGS. 1A-1B) while the second container 140 (FIGS. 1A-1B) is coupled to the second end 106 (FIGS. 1A-1B) of the first container 102 (FIGS. 1A-1B). In particular, block 610 can include inserting the first food into the first container 102 (FIGS. 1A-1B) through the lid 116 (FIGS. 1A-1B) while the flap 128 (FIGS. 1A-1B) is in an open position.

In a number of embodiments, the method 600 further can include inserting a first supplement into the first container (block 612). For example, block 612 can include inserting a first supplement (e.g., calcium, vitamin, etc.) into the first container 102 (FIGS. 1A-1B) through the first end 104 (FIGS. 1A-1B) of the first container 102 (FIGS. 1A-1B) while the second container 140 (FIGS. 1A-1B) is coupled to the second end 106 (FIGS. 1A-1B) of the first container 102 (FIGS. 1A-1B). In particular, block 612 can include inserting the first supplement into the first container 102 (FIGS. 1A-1B) through the lid 116 (FIGS. 1A-1B) while the flap 128 (FIGS. 1A-1B) is in an open position. In some embodiments, the first supplement is inserted into the first container 102 (FIGS. 1A-1B) from the third container 160 (FIGS. 1A-1B). In some embodiments, block 612 is performed before block 610, or both before and after block 610.

In a number of embodiments, the method 600 additionally can include closing the first lid before mixing the first food and the first supplement (block 614). For example, block 614 can include closing the lid 116 (FIGS. 1A-1B) before mixing the first food and the first supplement together in the first container 102 (FIGS. 1A-1B). In particular, block 614 can include moving the flap 128 (FIGS. 1A-1B) of the lid 116 (FIGS. 1A-1B) into the closed portion.

In a number of embodiments, the method 600 also can include mixing the first food and the first supplement (block 616). For example, block 616 can include mixing the first food and the first supplement together in the first container 102 (FIGS. 1A-1B) to coat the first food with the first supplement, such that an excess amount of the first supplement passes through the second end 106 (FIGS. 1A-1B) of the first container 102 (FIGS. 1A-1B) onto the second lid 158 (FIGS. 1A-1B) coupled to the second container 140 (FIGS. 1A-1B), which is coupled to the second end 106 (FIGS. 1A-1B) of the first container 102 (FIGS. 1A-1B). As an example, the mixing can be performed by shaking first container 102 (FIGS. 1A-1B).

In a number of embodiments, block 616 can include keeping the first lid closed while mixing the first food and the first supplement (block 618). For example, block 618 can include keeping the lid 116 (FIGS. 1A-1B) closed while mixing the first food and the first supplement together in the first container 102 (FIGS. 1A-1B). In particular, block 618 can include keeping the flap 128 (FIGS. 1A-1B) of the lid 116 (FIGS. 1A-1B) in the closed position while mixing the first food and the first supplement.

In a number of embodiments, the method 600 additionally can include opening the first lid after mixing the first food and the first supplement together in the first container and before removing the first food, as coated with the first supplement, from the first container (block 620). For example, block 620 can include opening the lid 116 (FIGS. 1A-1B) after mixing the first food and the first supplement together in the first container 102 (FIGS. 1A-1B) and before removing the first food, as coated with the first supplement, from the first container 102 (FIGS. 1A-1B). In these embodiments, the flap 128 (FIGS. 1A-1B) of the lid 116 (FIGS. 1A-1B) can be moved into the open position. In other embodiments, opening the first lid can include removing the lid 116 (FIGS. 1A-1B) from the first container 102 (FIGS. 1A-1B) by decoupling the lid 116 (FIGS. 1A-1B) from the first end 104 (FIGS. 1A-1B) of the first container 102 (FIGS. 1A-1B).

In a number of embodiments, the method 600 also can include removing the first food, as coated with the first supplement, from the first container (block 622). For example, block 622 can include removing the first food, as coated with the first supplement, from the first container 102 (FIGS. 1A-1B). In particular, block 622 can include removing the first food, as coated with the first supplement, from the first container through the lid 116 (FIGS. 1A-1B) when the flap 128 (FIGS. 1A-1B) is in the open position. In some embodiments, a tool (e.g., tweezers, forceps, spoon, etc.) can be used to remove the first food, as coated with the first supplement, from the first container 102 (FIGS. 1A-1B), regardless of whether the lid 116 (FIGS. 1A-1B) remains coupled to the first container 102 (FIGS. 1A-1B).

In a number of embodiments, the method 600 further can include removing the second container from the second end of the first container (block 624). For example, block 624 can include removing the second container 140 (FIGS. 1A-1B) from the second end 106 (FIGS. 1A-1B) of the first container 102 (FIGS. 1A-1B) with the excess amount of the first supplement on the second lid 158 (FIGS. 1A-1B) that passed through the structure 200 (FIG. 2A) when the first food and the first supplement were being mixed in the first container 102 (FIGS. 1A-1B). In some embodiments, the removing can include screwing or twisting the second container 140 (FIGS. 1A-1B) off of the second end 106 (FIGS. 1A-1B) of the first container 102 (FIGS. 1A-1B). In other embodiments, the removing can include undoing a snap-on coupling, a smooth friction fit, or the like.

In a number of embodiments, the method 600 additionally can include disposing the excess amount of the first supplement from the second lid into a third container (block 626). For example, block 626 can include disposing the excess amount of the first supplement that passed through the structure 200 (FIG. 2A) onto the second lid 158 (FIGS. 1A-1B) into the third container 160 (FIGS. 1A-1B).

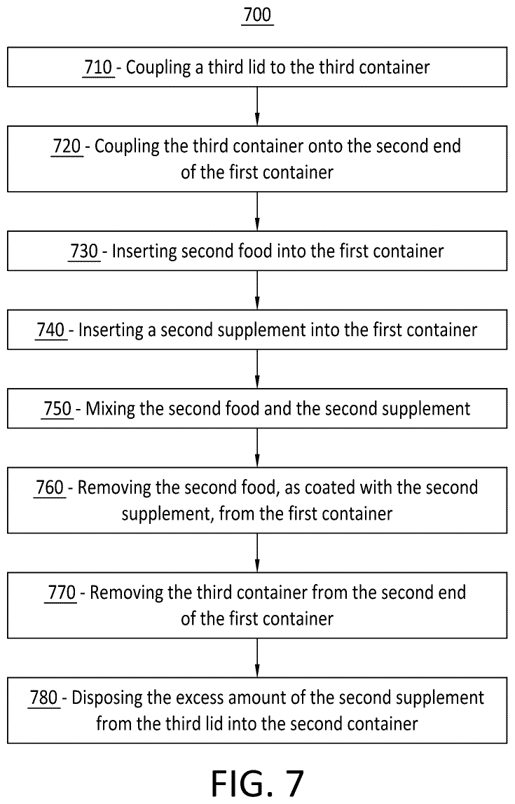

Turning ahead in the drawings, FIG. 7 illustrates a flowchart of a method 700 for using a supplement container (e.g., 100 (FIGS. 1A-1B)), according to an embodiment. As an example, method 700 can be used to coat food. In a number of embodiments, the supplement container of method 700 can be similar or identical to the supplement container 100 (FIGS. 1A-1B). In some embodiments, the method 700 can be performed after the method 600 (FIG. 6).

In a number of embodiments, the method 700 can include coupling a third lid to a third container (block 710). For example, block 710 can include coupling the third lid 166 (FIGS. 1A-1B) to the third container 160 (FIGS. 1A-1B). In some embodiments, the coupling can include pushing the third lid 166 (FIGS. 1A-1B) onto the third container 160 (FIGS. 1A-1B) to secure the third lid 166 (FIGS. 1A-1B) onto the third container 160 (FIGS. 1A-1B) via a smooth friction fit. In other embodiments, the coupling can include a snap-on coupling, a screwing or twisting motion, or the like.

In a number of embodiments, the method 700 also can include coupling the third container to the second end of the first container (block 720). For example, block 710 can include coupling the third container 160 (FIGS. 1A-1B) to the second end 106 (FIGS. 1A-1B) of the first container 102 (FIGS. 1A-1B) while the third lid 166 (FIGS. 1A-1B) is coupled to the third container 160 (FIGS. 1A-1B). In some embodiments, the coupling can include screwing or twisting the third container 160 (FIGS. 1A-1B) onto the second end 106 (FIGS. 1A-1B) of the first container 102 (FIGS. 1A-1B). In other embodiments, the coupling can include a snap-on coupling, a smooth friction fit, or the like.

In a number of embodiments, the method 700 further can include inserting second food into the first container (block 730). For example, block 730 can include inserting second food (e.g., insects, pet food, etc.) into the first container 102 (FIGS. 1A-1B) through the first end 104 (FIGS. 1A-1B) of the first container 102 (FIGS. 1A-1B) while the third container 160 (FIGS. 1A-1B) is coupled to the second end 106 (FIGS. 1A-1B) of the first container 102 (FIGS. 1A-1B). In particular, block 730 can include inserting the second food into the first container 102 (FIGS. 1A-1B) through the lid 116 (FIGS. 1A-1B) while the flap 128 (FIGS. 1A-1B) is in an open position.

In a number of embodiments, the method 700 additionally can include inserting a second supplement into the first container (block 740). For example, block 740 can include inserting a second supplement (e.g., calcium, vitamin, etc.) into the first container 102 (FIGS. 1A-1B) through the first end 104 (FIGS. 1A-1B) of the first container 102 (FIGS. 1A-1B) while the third container 160 (FIGS. 1A-1B) is coupled to the second end 106 (FIGS. 1A-1B) of the first container 102 (FIGS. 1A-1B). In particular, block 740 can include inserting the second supplement into the first container 102 (FIGS. 1A-1B) through the lid 116 (FIGS. 1A-1B) while the flap 128 (FIGS. 1A-1B) is in an open position. In some embodiments, the second supplement is inserted into the first container 102 (FIGS. 1A-1B) from the second container 140 (FIGS. 1A-1B). In some embodiments, block 740 is performed after block 730, or both before and after block 730.

In a number of embodiments, the method 700 also can include mixing the second food and the second supplement (block 750). For example, block 750 can include mixing the second food and the second supplement together in the first container 102 (FIGS. 1A-1B) to coat the second food with the second supplement, such that an excess amount of the second supplement passes through the second end 106 (FIGS. 1A-1B) of the first container 102 (FIGS. 1A-1B) onto the third lid 166 (FIGS. 1A-1B) coupled to the third container 160 (FIGS. 1A-1B), which is coupled to the second end 106 (FIGS. 1A-1B) of the first container 102 (FIGS. 1A-1B). In some embodiments, the mixing can include shaking first container 102 (FIGS. 1A-1B). In a number of embodiments, the method 700 can include closing the lid 116 (FIGS. 1A-1B) before mixing the second food and the second supplement together in the first container 102 (FIGS. 1A-1B). In particular, closing the lid 116 (FIGS. 1A-1B) can include moving the flap 128 (FIGS. 1A-1B) of the lid 116 (FIGS. 1A-1B) into the closed portion.

In a number of embodiments, the method 700 further can include removing the second food, as coated with the second supplement, from the first container (block 760). For example, block 760 can include removing the second food, as coated with the second supplement, from the first container 102 (FIGS. 1A-1B). In particular, block 760 can include removing the second food, as coated with the second supplement, from the first container 102 (FIGS. 1A-1B) through the lid 116 (FIGS. 1A-1B) when the flap 128 (FIGS. 1A-1B) is in the open position. In some embodiments, a tool (e.g., tweezers, forceps, spoon, etc.) can be used to remove the second food, as coated with the second supplement, from the first container 102 (FIGS. 1A-1B), regardless of whether the lid 116 (FIGS. 1A-1B) remains coupled to the first container 102 (FIGS. 1A-1B).

In a number of embodiments, the method 700 additionally can include removing the third container from the second end of the first container (block 770). For example, removing the third container 160 (FIGS. 1A-1B) from the second end 106 (FIGS. 1A-1B) of the first container 102 (FIGS. 1A-1B) with the excess amount of the second supplement on the third lid 166 (FIGS. 1A-1B) that passed through the structure 200 (FIG. 2A) when the second food and the second supplement were being mixed in the first container 102 (FIGS. 1A-1B). In some embodiments, the removing can include screwing or twisting the third container 160 (FIGS. 1A-1B) off of the second end 106 (FIGS. 1A-1B) of the first container 102 (FIGS. 1A-1B). In other embodiments, the removing can include undoing a snap-on coupling, a smooth friction fit, or the like.

In a number of embodiments, the method 700 also can include disposing the excess amount of the second supplement from the third lid into the second container (block 780). For example, block 780 can include disposing the excess amount of the second supplement that passed through the structure 200 (FIG. 2A) onto the third lid 166 (FIGS. 1A-1B) into the second container 140 (FIGS. 1A-1B).

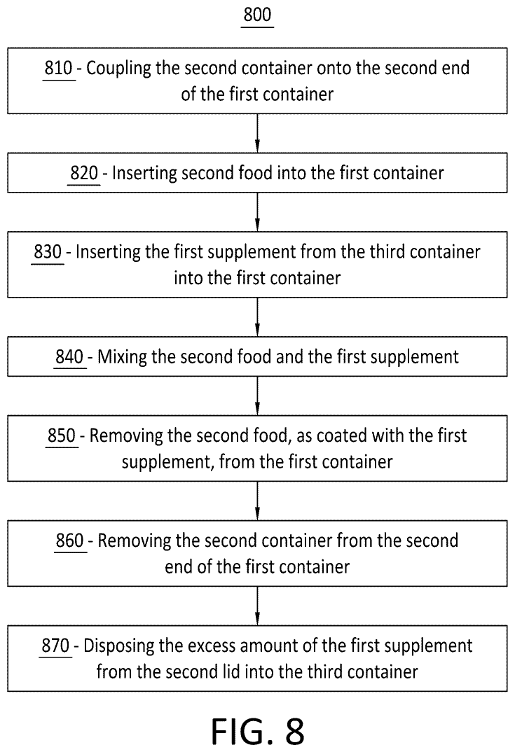

Turning ahead in the drawings, FIG. 8 illustrates a flowchart of a method (800) for using a supplement container (e.g., 100 (FIGS. 1A-1B)), according to an embodiment. As an example, method 800 can be used to coat food. In a number of embodiments, the supplement container of method 800 can be similar or identical to the supplement container 100 (FIGS. 1A-1B). In some embodiments, the method 800 can be performed after the method 600 (FIG. 6), and can be performed before or after method 700 (FIG. 7).

In a number of embodiments, the method 800 can include coupling the second container to the second end of the first container (block 810). For example, block 810 can include coupling the second container 140 (FIGS. 1A-1B) to the second end 106 (FIGS. 1A-1B) of the first container 102 (FIGS. 1A-1B) while the second lid 158 (FIGS. 1A-1B) is coupled to the second container 140 (FIGS. 1A-1B). In some embodiments, the coupling can include screwing or twisting the second container 140 (FIGS. 1A-1B) onto the second end 106 (FIGS. 1A-1B) of the first container 102 (FIGS. 1A-1B). In other embodiments, the coupling can include a snap-on coupling, a smooth friction fit, or the like.

In a number of embodiments, the method 800 also can include inserting second or third food into the first container (block 820). For example, block 820 can include inserting second or third food (e.g., insects, pet food, etc.) into the first container 102 (FIGS. 1A-1B) through the first end 104 (FIGS. 1A-1B) of the first container 102 (FIGS. 1A-1B) while the second container 140 (FIGS. 1A-1B) is coupled to the second end 106 (FIGS. 1A-1B) of the first container 102 (FIGS. 1A-1B). In particular, block 820 can include inserting the second or third food into the first container 102 (FIGS. 1A-1B) through the lid 116 (FIGS. 1A-1B) while the flap 128 (FIGS. 1A-1B) is in an open position.

In a number of embodiments, the method 800 further can include inserting the first supplement from the third container into the first container (block 830). For example, block 830 can include inserting the first supplement (e.g., calcium, vitamin, etc.) into the first container 102 (FIGS. 1A-1B) through the first end 104 (FIGS. 1A-1B) of the first container 102 (FIGS. 1A-1B) while the second container 140 (FIGS. 1A-1B) is coupled to the second end 106 (FIGS. 1A-1B) of the first container 102 (FIGS. 1A-1B). In particular, block 830 can include inserting the first supplement into the first container 102 (FIGS. 1A-1B) through the lid 116 (FIGS. 1A-1B) while the flap 128 (FIGS. 1A-1B) is in an open position. In some embodiments, block 830 is performed after block 820, or both before and after block 820. In the same or different embodiments, the first supplement is inserted into the first container 102 (FIGS. 1A-1B) from the third container 160 (FIGS. 1A-1B). In some embodiments where method 800 is performed after method 600 (FIG. 6) and the first supplement is inserted into the first container 102 (FIGS. 1A-1B) from the third container 160 (FIGS. 1A-1B), the first supplement from the third container 160 (FIGS. 1A-1B) might be only the excess amount of the first supplement that was disposed into the third container 160 (FIGS. 1A-1B) in block 626 (FIG. 6), and in other embodiments, the first supplement from the third container 160 (FIGS. 1A-1B) include the excess amount of the first supplement that was disposed into the third container 160 (FIGS. 1A-1B) in block 626 (FIG. 6), plus an additional amount of the first supplement, and in still further embodiments, the first supplement from the third container 160 (FIGS. 1A-1B) does not include any of the excess amount of the first supplement that was disposed into the third container 160 (FIGS. 1A-1B) in block 626 (FIG. 6).

In a number of embodiments, the method 800 additionally can include mixing the second or third food and the first supplement (block 840). For example, block 840 can include mixing the second or third food and the first supplement together in the first container 102 (FIGS. 1A-1B) to coat the second or third food with the first supplement, such that an excess amount of the first supplement passes through the second end 106 (FIGS. 1A-1B) of the first container 102 (FIGS. 1A-1B) onto the second lid 158 (FIGS. 1A-1B) coupled to the second container 140 (FIGS. 1A-1B), which is coupled to the second end 106 (FIGS. 1A-1B) of the first container 102 (FIGS. 1A-1B). In some embodiments, the mixing can include shaking first container 102 (FIGS. 1A-1B). In a number of embodiments, the method 800 can include closing the lid 116 (FIGS. 1A-1B) before mixing the second or third food and the first supplement together in the first container 102 (FIGS. 1A-1B). In particular, closing the lid 116 (FIGS. 1A-1B) can include moving the flap 128 (FIGS. 1A-1B) of the lid 116 (FIGS. 1A-1B) into the closed portion.

In a number of embodiments, the method 800 also can include removing the second or third food, as coated with the first supplement, from the first container (block 850). For example, block 850 can include removing the second or third food, as coated with the first supplement, from the first container 102 (FIGS. 1A-1B). In particular, block 850 can include removing the second or third food, as coated with the first supplement, from the first container 102 (FIGS. 1A-1B) through the lid 116 (FIGS. 1A-1B) when the flap 128 (FIGS. 1A-1B) is in the open position. In some embodiments, a tool (e.g., tweezers, forceps, spoon, etc.) can be used to remove the second or third food, as coated with the second supplement, from the first container 102 (FIGS. 1A-1B), regardless of whether the lid 116 (FIGS. 1A-1B) remains coupled to the first container 102 (FIGS. 1A-1B).

In a number of embodiments, the method 800 further can include removing the second container from the second end of the first container (block 860). For example, block 860 can include removing the second container 140 (FIGS. 1A-1B) from the second end 106 (FIGS. 1A-1B) of the first container 102 (FIGS. 1A-1B) with the excess amount of the first supplement on the second lid 158 (FIGS. 1A-1B) that passed through the structure 200 (FIG. 2A) when the second or third food and the first supplement were being mixed in the first container 102 (FIGS. 1A-1B). In some embodiments, the removing can include screwing or twisting the second container 140 (FIGS. 1A-1B) off of the second end 106 (FIGS. 1A-1B) of the first container 102 (FIGS. 1A-1B). In other embodiments, the removing can include undoing a snap-on coupling, a smooth friction fit, or the like.

In a number of embodiments, the method 800 additionally can include disposing the excess amount of the first supplement from the second lid into the third container (block 870). For example, block 870 can include disposing the excess amount of the first supplement that passed through the structure 200 (FIG. 2A) onto the second lid 158 (FIGS. 1A-1B) into the third container 160 (FIGS. 1A-1B).

Although supplement containers and methods of use for animals have been described with reference to specific embodiments, it will be understood by those skilled in the art that various changes may be made without departing from the spirit or scope of the disclosure. Accordingly, the disclosure of embodiments is intended to be illustrative of the scope of the disclosure and is not intended to be limiting. It is intended that the scope of the disclosure shall be limited only to the extent required by the appended claims. For example, to one of ordinary skill in the art, it will be readily apparent that any components of the supplement container disclosed herein, as well as the steps to use the supplement container, may be modified, and that the foregoing discussion of certain of these embodiments does not necessarily represent a complete description of all possible embodiments. In some embodiments, the supplement containers and methods of use can be utilized in bee mite inspection/care. For example, bees can be placed in the main container along with powdered sugar and shaken gently so that the powdered sugar and any mites pass into the supplement container for inspection. In some embodiments, the main container can be filled with a food item (e.g., vegetables, meats, animal food, etc.) and a seasoning and shaken to coat the food item with the seasoning.

Replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

What is claimed is:

1. An apparatus comprising:
a first container comprising a first end and a second end, wherein the first end comprises a first fastener located on a first surface of the first container and the second end comprises a second fastener positioned on a second surface of the first container, and wherein the first surface is different form the second surface;
a first lid comprising a third end and a fourth end, wherein the fourth end comprises a third fastener located on a surface of the first lid, wherein the first lid is configured to be coupled to the first end of the first container by coupling together the third fastener and the first fastener; and
a second container including a fifth end and a sixth end, wherein the fifth end comprises a fourth fastener located on a third surface of the second container, wherein the sixth end comprises a fifth fastener located on a fourth surface of the second container, wherein the third surface is different from the fourth surface, and wherein the second container is configured to be coupled to the second end of the first container by coupling together the fourth fastener and the second fastener.

2. The apparatus of claim 1, wherein the second end of the first container comprises a structure comprising apertures.

3. The apparatus of claim 2, wherein:
the second container further comprises walls defining a cavity;
the second container further comprises a second lid sized to create a friction fit with the walls to seal the cavity; and
when the second lid is not sealing the cavity and when the second container is coupled to the second end of the first container, contents within the first container can pass through the apertures of the structure to enter the cavity.

4. The apparatus of claim 1, wherein the first container is comprised of a transparent static-free material.

5. The apparatus of claim 4, wherein the first lid and the second container are comprised of the transparent static-free material.

6. The apparatus of claim 1, wherein the first fastener of the first container and the third fastener of the first lid comprise threaded fasteners that couple together with each other to couple together the first lid and the first container.

7. The apparatus of claim 1, wherein:
the third end of the first lid comprises a first protrusion, a flap comprising a second protrusion, and a hinge;
the flap pivots about the hinge to allow access to an internal portion of the first container when the first lid is coupled to the first container; and
the first protrusion and the second protrusion are located and sized to create a snap-fit connection with each other to mitigate the flap from moving during operation of the apparatus.

8. The apparatus of claim 1, wherein:
the first container is configured to contain food for an animal;
the second container is configured to contain at least one of a vitamin or a calcium supplement; and
the at least one of the vitamin or the calcium supplement can pass from the second container into the first container to coat the food.

9. The apparatus of claim 1, further comprising a third container adapted to be coupled to the sixth end of the second container.

10. The apparatus of claim 9, wherein:
the third container comprises a recess on an external surface of the third container; and
the recess is adapted to receive a label.

11. A method of manufacturing an apparatus, the method comprising:
providing a first container, the first container comprising a first end and a second end, wherein the first end comprises a first fastener located on a first surface of the first container and the second end comprises a second fastener positioned on a second surface of the first container, and wherein the first surface is different form the second surface;
providing a first lid, the first lid comprising a third end and a fourth end, wherein the fourth end comprises a third fastener located on a surface of the first lid, wherein the first lid is configured to be coupled to the first end of the first container by coupling together the third fastener and the first fastener; and
providing a second container, the second container including a fifth end and a sixth end, wherein the fifth end comprises a fourth fastener located on a third surface of the second container, wherein the sixth end comprises a fifth fastener located on a fourth surface of the second container, wherein the third surface is different from the fourth surface, and wherein the second container is configured to be coupled to the second end of the first container by coupling together the fourth fastener and the second fastener.

12. The method of claim 11, further providing a third container, the third container configured to be coupled to the sixth end of the second container.

13. The method of claim 11, wherein providing the first container further comprises providing a structure comprising apertures on the second end of the first container.

14. The method of claim 13, wherein providing the second container further comprises providing walls defining a cavity in the second container, and wherein the method further comprises providing a second lid sized to create a friction fit with the walls to seal the cavity.

15. The method of claim 11, wherein the first fastener of the first container and the third fastener of the first lid comprise threaded fasteners that couple together with each other to couple together the first lid and the first container.

16. A method of using a supplement container, the method comprising:
coupling a second lid to a second container;
coupling the second container to a second end of a first container while the second lid is coupled to the second container;
inserting first food into the first container through a first end of the first container while the second container is coupled to the second end of the first container;
inserting a first supplement into the first container through the first end of the first container while the second container is coupled to the second end of the first container;
mixing the first food and the first supplement together in the first container to coat the first food with the first supplement, such that an excess amount of the first supplement passes through the second end of the first container onto the second lid coupled to the second container, which is coupled to the second end of the first container;
removing the first food, as coated with the first supplement, from the first container; and
removing the second container from the second end of the first container with the excess amount of the first supplement on the second lid.

17. The method of claim 16, further comprising:
coupling a third lid onto a third container;
coupling the third container onto the second end of the first container while the third lid is coupled to the third container;
inserting second food into the first container through the first end of the first container while the third container is coupled to the second end of the first container;
inserting a second supplement into the first container through the first end of the first container while the third container is coupled to the second end of the first container;
mixing the second food and the second supplement together in the first container to coat the second food with the second supplement, such that an excess amount of the second supplement passes through the second end of the first container onto the third lid coupled to the third container, which is coupled to the second end of the first container;
removing the second food, as coated with the second supplement, from the first container; and
removing the third container from the second end of the first container with the excess amount of the second supplement on the third lid.

18. The method of claim 16, further comprising:
disposing the excess amount of the first supplement from the second lid into a third container;
coupling the second container onto the second end of the first container while the second lid is coupled to the second container;
inserting second food into the first container through the first end of the first container while the second container is coupled to the second end of the first container;
inserting the excess amount of the first supplement from the third container into the first container through the first end of the first container while the second container is coupled to the second end of the first container;
mixing the second food and the excess amount of the first supplement together in the first container to coat the second food with the first supplement, such that an excess amount of the excess amount of the first supplement passes through the second end of the first container onto the second lid coupled to the second container, which is coupled to the second end of the first container;
removing the second food, as coated with the excess amount of the first supplement, from the first container; and
removing the second container from the second end of the first container with the excess amount of the excess amount of the first supplement on the second lid.

19. The method of claim 16, further comprising:
coupling a first lid to the first end of the first container; and
opening the first lid before inserting the first food into the first container,
wherein:
inserting the first food into the first container comprises inserting the first food into the first container through the first lid.

20. The method of claim 19, further comprising:
closing the first lid before mixing the first food and the first supplement together in the first container; and
opening the first lid after mixing the first food and the first supplement together in the first container and before removing the first food, as coated with the first supplement, from the first container,
wherein:
mixing the first food and the first supplement together in the first container comprises keeping the first lid closed while mixing the first food and the first supplement together in the first container; and
removing the first food, as coated with the first supplement, from the first container comprises removing the first food, as coated with the first supplement, from the first container through the first lid.
